(19) United States
(12) Patent Application Publication
(10) Pub. No.: US 2025/0276164 A1
(43) Pub. Date: Sep. 4, 2025

(54) BIO-INSPIRED NEEDLE FOR CONTROLLED RELEASE

(71) Applicant: THE REGENTS OF THE UNIVERSITY OF CALIFORNIA, Oakland, CA (US)

(72) Inventors: Yazhi SUN, La Jolla, CA (US); Xiaocheng WANG, La Jolla, CA (US); Shaochen CHEN, San Diego, CA (US)

(21) Appl. No.: 19/067,860

(22) Filed: Mar. 1, 2025

Related U.S. Application Data
(60) Provisional application No. 63/560,407, filed on Mar. 1, 2024.

(51) Int. Cl. A61M 37/00; A61L 31/02; A61L 31/04; A61L 31/14; A61L 33/00

(52) U.S. Cl. CPC A61M 37/0015; A61L 31/024; A61L 31/041; A61L 31/145; A61L 33/0011; A61L 2300/236; A61L 2400/06; A61M 2037/0023; A61M 2205/02

(57) ABSTRACT

A micro-needle (MN) patch is formed as an array of 3-D bioprinted structures having a relatively hard planar base with multiple water sensitive barbed tips extending therefrom. The barbed tips are formed from a polymer material configured to exhibit reversible shrink-swell volume change in response to surrounding humidity to facilitate skin penetration under the shrinkage conditions and long-term adhesion when swelling in skin tissues.

### RELATED APPLICATIONS

This application claims the benefit of the priority of Application No. 63/560,407, filed Mar. 1, 2024, which is incorporated herein by reference in its entirety.

### GOVERNMENT RIGHTS

This invention was made with government support under Grant Nos. 2025752 and 2135720 awarded by the National Science Foundation (NSF). The government has certain rights in the invention.

### BACKGROUND

Controllable and long-term release remains a great challenge in current drug delivery systems. Benefiting from their efficient drug loading and painless administration, microneedles (MNs) have emerged as a promising platform for transdermal drug delivery, however they often fail to achieve long-term tissue adhesion and controllable extended drug release.

Controlled drug release systems have shown significant promise in clinical applications, primarily based on their ability to maintain sustainable drug concentrations during administration. This approach helps prevent drug overdose resulting from repeated administration in addition to facilitating patient compliance. Currently, widely-used drug delivery systems, such as implantable pumps, electrospun nanofibers, and nanoparticles, encounter the challenge of initial burst release due to the fact that their release mechanisms largely rely on drug molecule diffusion or material degradation. Various strategies, including creating artificial gradients, fine-tuning material degradability or diffusion coefficients, and modifying nanoparticle surfaces, have been explored to extend drug release duration. Although these solutions have provided some improvement in the long-term release behavior of drug delivery systems, they usually necessitate repeated implantation surgeries or injections, resulting in patient discomfort and reduced treatment adherence. By contrast, microneedles (MNs) offer transdermal delivery, requiring only minimal skin penetration to minimize nerve stimulation, providing a painless, or nearly painless, method. Unfortunately, traditional microneedles with simple flat side profiles suffer from limited adhesion to the skin. In more recent developments, MNs have been reported with nature-inspired microstructures to enhance long-term adhesion in tissues. For example, MNs have been designed with backward barbs modeled on honey bee and mosquito stingers. (Sec, e.g., Tran, L.-G.; et al., 2019 IEEE 32nd Int'l Conf. on MEMS, pp 547-550; Lu, Y., et al., Acta Biomater. 2022:153, pp. 386-398; Cai, Y., et al., Research 2022.) While these bio-inspired tip structures have exhibited stronger tissue adhesion capacity, they have been unable to maintain sustainable drug release for extended periods due to their reliance on physical diffusion of drug molecules. In these designs, material degradation can result in almost complete drug release within one day or even a few hours. Accordingly, the need remains for innovative MNs with hierarchical microstructures and controllable long-term release capability for effective drug delivery systems.

### SUMMARY

Embodiments disclosed herein relate to a three-dimensional (3D) printed microneedle (MN) patch with succulent-inspired responsive microstructures and light-controllable long-term release capability. The inventive MN incorporates a reversible shrink-swell volume change behavior in response to surrounding humidity, which provides sufficient mechanical strength for skin penetration under the shrinkage conditions and efficient long-term adhesion when swollen in skin tissues. Moreover, the MN patch introduces a controllable long-term drug release system, achieved through the integration of thiolated heparin (Hep-SH) for sustained growth factor release and graphene oxide (GO) nanosheets for controlled drug release via near infrared (NIR) laser irradiation. MN patches fabricated according the methods described herein were evaluated for use in growth factor loading, exhibiting good biocompatibility and effectively promoting the proliferation, migration, and proangiogenesis of endothelial cells. Such flexible MN patches provide a solution for controllable long-term transdermal drug delivery as well as other related tissue engineering applications.

In one aspect, a microneedle (MN) patch includes a 3-D bioprinted structure comprising: a substantially planar base formed from a first photocrosslinkable hydrogel material; and water-sensitive barbed structures formed from a second photocrosslinkable hydrogel material printed onto and extending substantially perpendicular from a surface of the base, the barbed structures each having a volume configured to reversibly shrink and swell in response to surrounding humidity, wherein the barbed structures have a hardness configured to penetrate live skin tissue. The first photocrosslinkable hydrogel material may include a polyethylene glycol diacrylate (PEGDA) gel, and in some embodiments, may include graphene oxide (GO), wherein the GO is configured to heat in response to exposure to near infrared (NIR) light. The second photocrosslinkable hydrogel material may include a mixture of PEGDA and methacrylated hyaluronic acid (HAMA). In some embodiments, this mixture may be from 2.5% to 7.5% HAMA and from 7.5% to 20% PEGDA, and may be 7.5% HAMA and 7.5% PEGDA. The second photocrosslinkable hydrogel material may further thiolated heparin (Hep-SH) and/or a drug configured for transdermal delivery. The barbed structures may be printed in layers to define alternating layers of radially-extending barbs along a height of each barbed structure

The first photocrosslinkable hydrogel material and the second photocrosslinkable hydrogel material are configured to polymerize in response to exposure to light within a wavelength range of 365 nm to 405 nm.

In another aspect, a microneedle (MN) patch for transdermal drug delivery includes a substantially planar base 3-D bioprinted from a first bioink solution; and water-sensitive barbed structures 3-D bioprinted from a second bioink solution onto and extending substantially perpendicular from a surface of the base, the barbed structures each having a volume configured to reversibly shrink and swell in response to surrounding humidity, wherein the barbed structures have a hardness configured to penetrate live skin tissue. The first bioink solution may include a polyethylene glycol diacrylate (PEGDA) gel. The first bioink solution may further include graphene oxide (GO), wherein the GO is configured to heat in response to exposure to near infrared (NIR) light. The second bioink solution may include a mixture of PEGDA and methacrylated hyaluronic acid (HAMA). This mixture may be from 2.5% to 7.5% HAMA and from 7.5% to 20% PEGDA, and may be 7.5% HAMA and 7.5% PEGDA. The second bioink solution may further include one or more of thiolated heparin (Hep-SH) and a drug for transdermal delivery

The barbed structures may be printed in layers to define alternating layers of radially-extending barbs along a height of each barbed structure.

The first bioink solution and the second bioink solution are each configured to polymerize in response to exposure to light within a wavelength range of 365 nm to 405 nm.

### BRIEF DESCRIPTION OF THE DRAWINGS

FIG. 1 is a schematic illustration of the 3D printed MN patch with succulent-inspired responsive microstructures and light-controllable long-term release capability. The printed MN patches contained a relative hard base constructed from polyethylene glycol diacrylate (PEGDA) and a water sensitive tip composed of PEGDA and methacrylated hyaluronic acid (HAMA). As such, the MN patches have sufficient mechanical strength for skin penetration under the shrinkage conditions and efficient long-term adhesion when swollen in skin tissues. Moreover, the MN patch introduces a controllable long-term drug release system, achieved through the integration of thiolated heparin (Hep-SH) for sustained growth factor release and graphene oxide (GO) nanosheets for controlled drug release via near infrared (NIR) laser irradiation.

FIGS. 2A-2I illustrate aspects of morphology characterization of embodiments of succulent-inspired MN patches. FIG. 2A provides a 3D model of an exemplary MN design; FIG. 2B is a photograph of a fresh-printed MN patch on the printer probe; FIGS. 2C and 2D are photomicrographs of swollen and shrunken MN patches, respectively. The scale bar is 2 mm. FIGS. 2E-2F are fluorescent images of the sideview of fresh printed MN patches where the scale bars are 1 mm and 500 μm, respectively. FIG. 2G is a 3D construction of the MN patch from confocal microscopy; FIGS. 2H and 2I are top and side views SEM images, respectively.

FIGS. 3A-3D is a series of optical images of the MN patches, where FIG. 3A is side view; FIG. 3B is top view of a fresh-printed patch; FIG. 3C is a side view; and FIG. 3D is top view of a dehydrated MN patch. Scale bars are 2 mm in FIGS. 3A and 3C and 1 mm in FIGS. 3B and 3D.

FIG. 4 is a graph showing printability of the prepolymer bioinks with varying PEGDA and HA contents.

FIGS. 5A-5F illustrate composition optimization and mechanical properties, where FIG. 5A shows volume change of the MN patches when immersed in PBS; FIG. 5B shows volume change of the MN patches after reversible dehydration and hydration for 5 cycles; FIG. 5C plots degradation of the MN patches with different PEGDA contents and 7.5% HAMA; FIG. 5D shows compression force per needle with varied PEGDA contents; FIG. 5E illustrates maximum adhesion force of the MN patches on agarose gel (left) and chicken breast tissue (right) models; and FIG. 5F compares maximum adhesion force of the MN patches with different barb numbers on the agarose model. (*p<0.05, **p<0.01, ***p<0.001)

FIG. 6 provides digital photographs taken during the compression test. An MN sample was laid on a platform, where the MN tips pointed to the pressure sensor. The compression force was recorded as the sensor gradually moved to the tips. Arrows indicate the moving direction of the sensor.

FIG. 7 provides digital photographs taken during the adhesion tests on the agarose gel and chicken muscle tissue models. The tissue models were fixed on the bottom plate, while the MN sample was glued to the top customized sample holder, which was connected with a tensile sensor. The MN sample first penetrated into the tissue model and then was pulled out from the tissue models. The adhesion force was recorded as the sensor gradually moved away from the models.

FIGS. 8A-8F diagrammatically illustrate controllable drug release behaviors, where FIG. 8A is a series of photographs of the released RhoB from the MN patches into the chicken breast tissues over time; FIG. 8B plots the released RhoB contents in the chicken breast tissue over time; FIG. 8C is a plot of released BSA contents in PBS over time; FIG. 8D is a plot of real-time temperature change and corresponding thermal images (inset) under NIR irradiation (808 nm, 0.40 W/cm2, 5 min); FIG. 8E plots released BSA contents from different MN samples treated with or without NIR irradiation (808 nm, 0.40 W/cm2, 30 min) over time; and FIG. 8F is a plot of BSA release from different MN samples for 28 days. For the NIR groups, the MN samples were daily treated with NIR irradiation (808 nm, 0.40 W/cm2, 30 min).

FIG. 9 provides SEM images of the MN base and tips. Scale bar, 20 μm.

FIG. 10 plots photothermal heating curves of GO-incorporated MN samples under NIR irradiation with different power densities (GO content: 1 mg/mL, NIR: 808 nm, 5 min).

FIGS. 11A-11G diagrammatically illustrate in vitro compatibility and bioactivity of VEGF-loading MN patches, where FIG. 11A is a schematic illustration of the co-culture setting of the MN patches and HUVECs; FIG. 11B is a series of images of Calcein AM staining of HUVECs after 3 days of co-culture with different MN patches (scale bar=300 μm) and FIG. 11C provides the results of CCK8 assay of HUVECS co-cultured with different MN patches for 7 days; FIG. 11D shows an in vitro scratch assay of HUVECs co-cultured with different MN patches for 24 hours (scale bar=500 μm) and FIG. 11E plots the corresponding quantification of migrated area. FIG. 11F shows a tube formation assay of HUVECs after co-culture for 6 h (scale bar=300 μm) and FIG. 11G plots the corresponding quantification of the tube numbers. Cells were stained with Calcein AM. (In FIGS. 11C, 11C and 11G, *p<0.05, **p<0.01, ***p<0.001).

FIG. 12A plots ¹H NMR spectra of HA (bottom) and HAMA (top) confirming the successful methacrylation of HA.

FIG. 12B plots ¹H NMR spectra of Hep (heparin, bottom) and Hep-SH (top) confirming the successful thiolation of heparin.

### DETAILED DESCRIPTION OF EMBODIMENTS

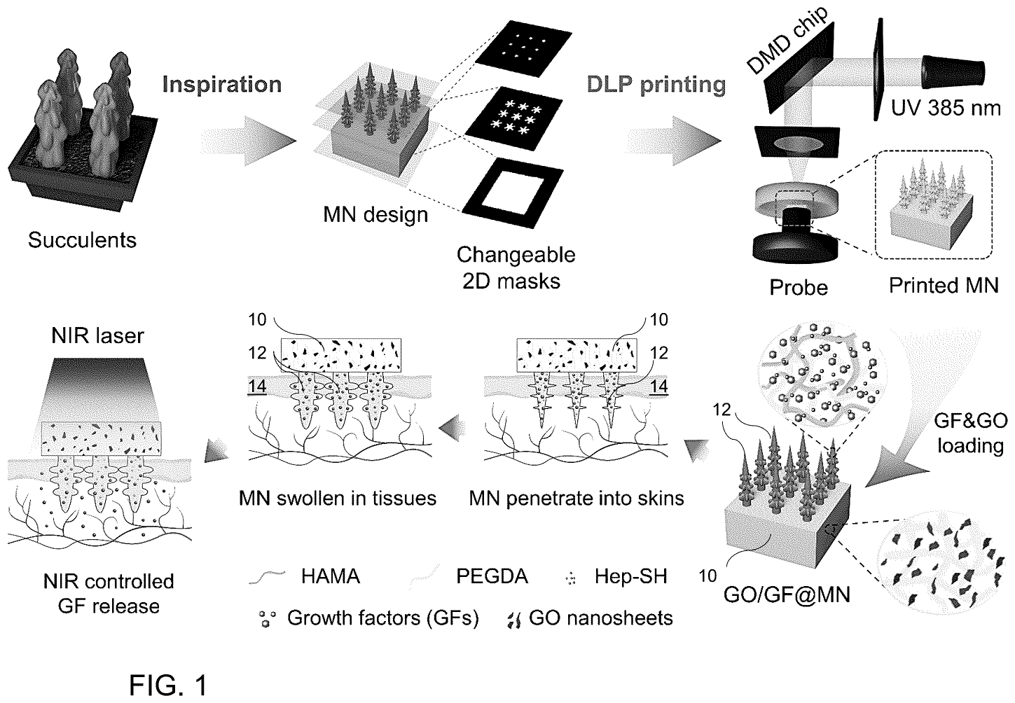

The inventive MN patch employs succulent-inspired responsive microstructures and light-controllable long-term release capability for transdermal drug delivery (FIG. 1). Succulents, in their evolutionary response to extreme draught conditions, have transitioned from the traditional leaf structure with a vast surface area to streamlined needle-like spines to reduce water loss from evaporation. The spines with oriented barbs enables succulents to collect water droplets from airborne moisture such as fog and absorb water through the interface between trichome and mucilage. Thus, succulents evolved to survive in arid conditions due to efficient water collection, relocation and storage enabled by these adaptative structures responding to variations in external water content.

For purposes of the present disclosure, the term “drug”, as in “drug-delivery”, broadly refers to a compound or “payload” that is configured and suitable for transdermal delivery, typically, but not exclusively, for therapeutic purposes. Thus, the term “drug” is intended to encompass, but is not limited to, pharmaceutical and medicinal compounds (synthetic or natural), e.g., small molecules (<1000 Daltons) and large molecules (>1000 Daltons), biologics, e.g., proteins, monoclonal antibodies, nucleic acids, growth factors, hormones, and supplements, including vitamins, minerals, and nutrients. Accordingly, the specific examples provided in the disclosure herein are for illustrative purposes and are not intended to be limiting.

The inventive hydrogel MN array features a reversible shrink-swell volume change behavior in response to surrounding humidity, providing sufficient mechanical strength for skin penetration under the shrinkage conditions and efficient long-term adhesion when swelling in skin tissues. In an important application as a drug-delivery mechanism, sustained long-term drug release, thiolated heparin (Hep-SH) has been demonstrated to extend the duration of drug release, because this polysaccharide contains dense negative charges, which is adept at entrapping positively charged common proteins such as growth factors. In addition, graphene oxide (GO) nanomaterials have attracted extensive interest in the construction of controllable drug delivery systems due to their unique photothermal properties and good biocompatibility. The incorporation of Hep-SH and GO into the hydrogel MN allows the succulent-inspired responsive microstructures to exhibit controllable long-term release capability for transdermal drug delivery.

As seen in the upper right panel of FIG. 1, a digital-light processing (DLP)-based 3D printing method can be employed to fabricate the MN patches with succulent-inspired barb structures and near infrared (NIR)-controlled long-term drug release capability. As an emerging technology in additive manufacture, DLP-based 3D printing empowers the efficient fabrication of complex 3D structures with fine features. Representative printing systems and methods that may be used or adapted to fabricate the inventive MN patches are disclosed in U.S. Pat. No. 11,833,742, and International Publication No. WO 204/118942, each of which is incorporated herein by reference.

The printed MN patches include a relatively hard base 10 formed from a first photocrosslinkable hydrogel material and an array of water sensitive tips 12 formed from a second photocrosslinkable hydrogel material that exhibits a reversible ability to swell or shrink in response to changes in external water content. It should be noted that while base 10 should be sufficiently hard and/or stiff to transfer application pressure to the tips for skin penetration, it should preferably also have a degree of flexibility to conform to and move with the skin surface after application.

In the test implementations, the first material used was polyethylene glycol diacrylate (PEGDA) while the second material used to form tips 12 was a mixture of PEGDA and methacrylated hyaluronic acid (HAMA). Alternative materials that may be employed for both the first and second materials by adapting the techniques disclosed herein include natural polymers such as methacrylated gelatin (GelMA), methacrylated alginate (AlgMA), and synthetic polymer such as acrylamide and N-isopropylacrylamide (NIPAM). Such materials are printable in the common wavelength range used in DLP bioprinting, i.e., from 365 nm to 405 nm. PEGDA in particular was selected for the present testing based on the relative case with which its mechanical properties could be modified through the addition of other crosslinkers such as N,N′ Methylenebis(acrylamide) (BIS).

When dehydrated, the inventive MN arrays possess sufficient mechanical strength to penetrate human skin while the MN tips 12 provide firm adhesion to the tissue for extended periods with the succulent-inspired barb structures hydrated and interlocked within the tissues. It should be noted that given the initial focus on development of a MN patch for human transdermal delivery, the tips described herein are designed to at least penetrate the stratum corneum (SC) which, in human skin, ranges in thickness from 10 to 30 μm. As will be apparent to those in the art, the thicknesses of other skin types, e.g., animal (non-human mammalian or reptilian), will determine the appropriate MN tip dimensions and characteristics to be used for MN patches for possible non-human applications.

Incorporation of Hep-SH in the MN tips facilitates the sustained release of growth factors by reducing the release speed, while embedded GO in the MN base accelerates drug release speed when exposed to NIR irradiation. This combination endows the MN patches with controllable long-term drug release capability. Additionally, experiments demonstrate that the MN patches were biocompatible, and that the incorporation of vascular endothelial growth factors (VEGF) into the MN patches can significantly promote the proliferation, migration, and proangiogenesis of cells, such as human umbilical vein endothelial cells (HUVECs), which have applications in immune response, wound healing, cellular response to viral or bacterial infection, oxidative stress, angiogenesis, arteriolosclerosis, drug screening, and tubule formation.

### Patch Fabrication and Morphology Characterization

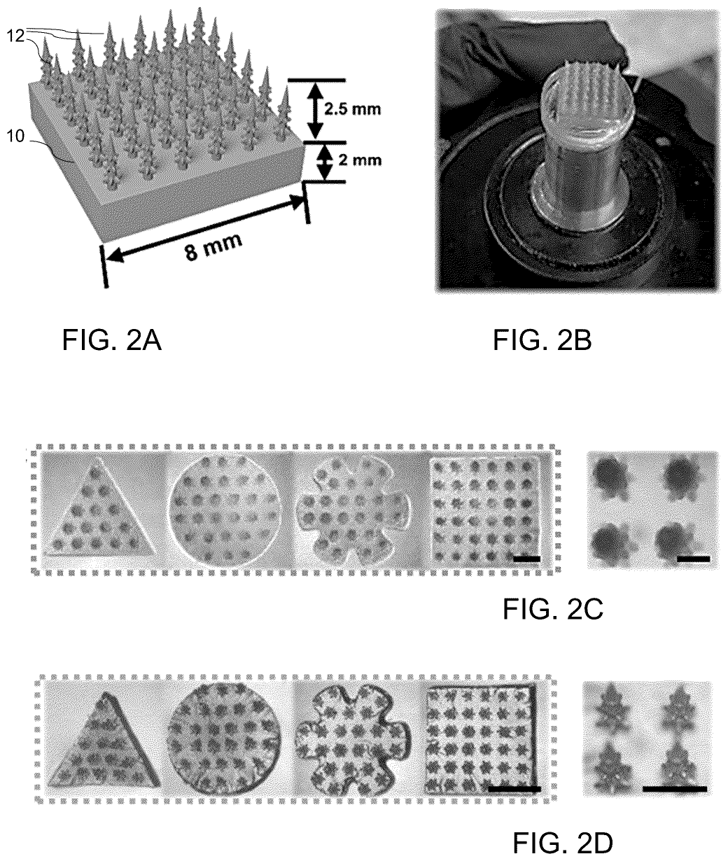

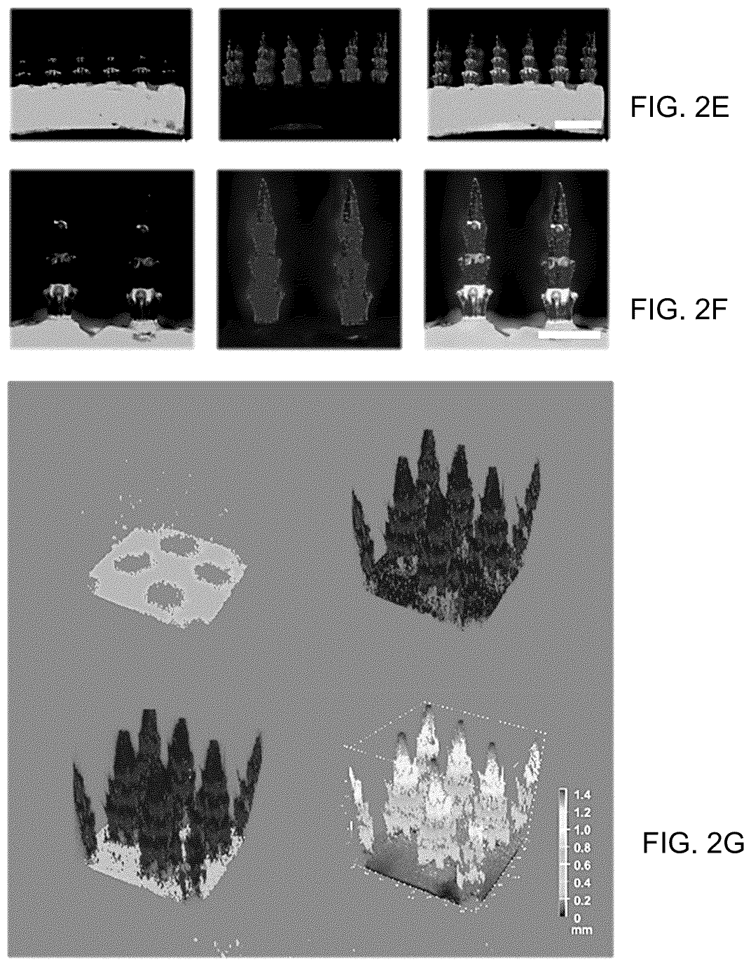

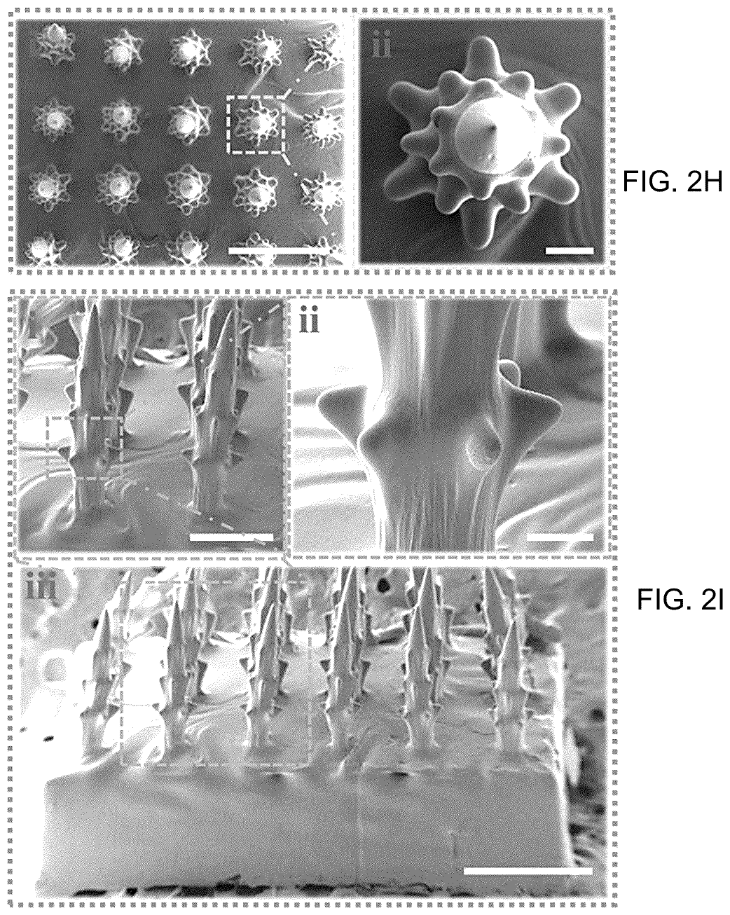

Referring to FIG. 2A, the succulent-inspired MN patch is configured as a relatively hard square base 10 with a dimension of 8 mm×8 mm×2 mm (length-width-height), with a shrinkable water-sensitive tip array 12 with a height of 2.5 mm. While a number of photocrosslinkable hydrogels are known in the art and may be used for fabrication, for evaluation, the biocompatible hydrogel PEGDA was selected for the base material due to its strong mechanical stiffness as compared to other prevalent photocrosslinkable hydrogels. To fabricate the MN tips, a material exhibiting a reliable reversible shrink-swell volume change is desired. This feature enables the MN tip to have sharp endpoints and the necessary mechanical strength to penetrate the skin after the dehydration of freshly printed hydrogel MN patches and allow multiple drug loadings and administrations. Once penetrated into the skin tissue, the MN tips 12 should be able to swell and expand by absorbing water, ensuring a stronger adhesion force and stable attachment. A combination of HAMA (a water-sensitive hydrogel) and PEGDA was selected for the tip material based on a combination of its reversible swell-shrink dynamics and in vivo biodegradation.

To fabricate an embodiment of the inventive structure, the 3D MN patch model was sliced into a stack of 2D masks along the vertical direction using a custom MATLAB® code. (Additional details are available in the previously-identified patent and patent publication.) Briefly, during the DLP printing process, light having a wavelength of 385 nm (LED, laser, or other light source) is reflected off of a digital micromirror device (DMD) chip and passed through a series of optical lenses to focus on a plane at which a petri-dish with polymer precursor was placed. (See, e.g., the diagram in the upper right panel of FIG. 1.) As the light pattern is projected onto the focal plane, the a precision motor is controlled by system controller to raise the printer probe at a user-defined speed, allowing a thin layer of the precursor solution between the probe and petri-dish to be polymerized with the designed pattern continuously along the vertical direction, to create intricate 3D structures. After printing the base 10 using PEGDA, another petri-dish containing a mixture of HAMA and PEGDA was positioned under the probe to continue the construction of the succulent-inspired tips 12 directly on the base 10 without requiring additional adhesion. A significant challenge in fabricating the tip portion is to control resolution of the overhanging barbs. During DLP printing, in addition to the photopolymerization occurring on the focal plane, light tended to diffuse or reflect to the precursor solution above the overhanging structure and scatter, leading to over-polymerization of the designed barbs. To mitigate this scattering effect, a photo absorber—tartrazine (0.04% w/v)—was included in the precursor solution. Tartrazine, a yellow food dye with the maximum absorption at 405 nm, is effective in enhancing the print quality and can be rinsed away after printing to leave transparent hydrogels.

FIG. 2B is a photograph of a freshly-printed MN patch, which was then immersed in phosphate-buffered saline (PBS) to remove tartrazine. Upon mild dehydration at 37° C., the MN patches shrank substantially isotropically to about half of their original size in diameter, preserving the detailed tip features. While the drawings primarily show a square base, the versatility of DLP printing allows a range of different base shapes to be fabricated to address a wide variety of clinical applications. FIGS. 2C and 2D provide non-limiting examples of possible base shapes, respectively showing the different shaped patches with swollen and shrunken tips.

Confocal microscopy was employed to examine the morphology of the inventive MN patch samples. With embedded fluorescence microspheres, the base part exhibited green fluorescence, whereas the tip array exhibited red fluorescence (FIG. 2E). The tip portion exhibited well-defined 3-layer extruding barbs, shown in FIG. 2F. The 3D reconstruction of the MN patches showed the height of tips to be around 1.4 mm, as seen in the lower right corner of FIG. 2G. Scanning electron microscope (SEM) showed the top view of alternating barbs from three layers forming a star-like structure, which can clearly be seen in the magnified section in the right panel of FIG. 2H. Scale bars are 1 mm in I (left image) and, 100 μm in ii (right image). FIG. 2I provides different magnifications (i, ii, and iii) of side views of the barbs with a length between 20 and 60 μm, showing a succulent-like microstructures. Scale bars represent 500 μm in i, 100 μm in ii, and 1 mm in iii.

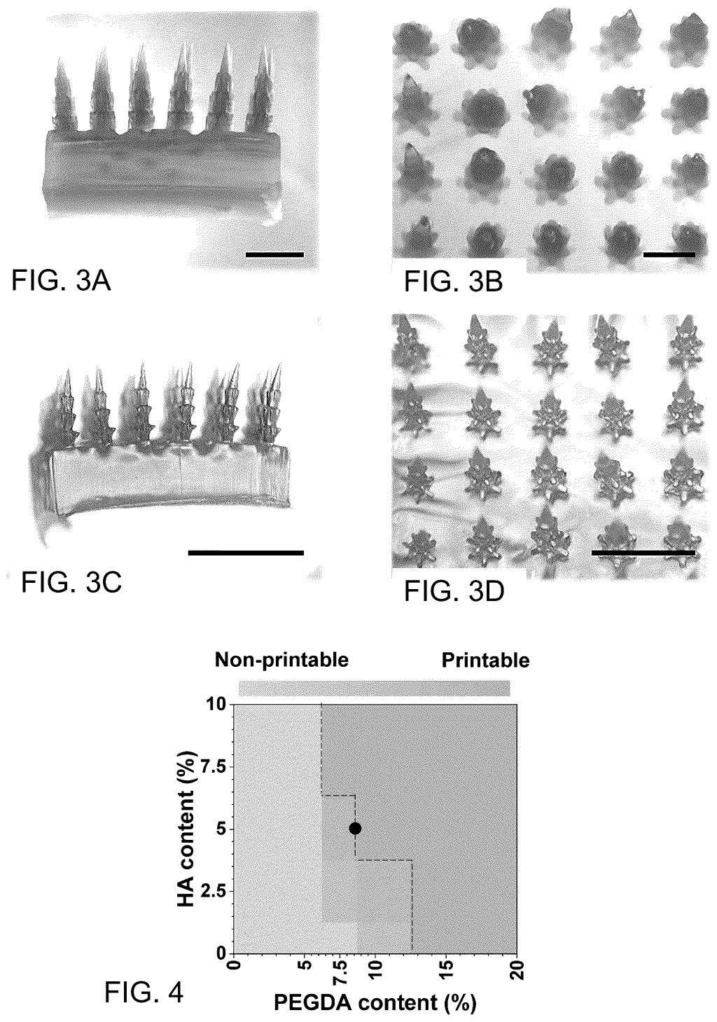

FIGS. 3A-3D provide optical images of exemplary freshly printed MN patches. FIGS. 3A and 3C are side views in which the scale bars are 2 mm. FIG. 3B and FIG. 3D are both top views, where the patch in FIG. 3D is dehydrated. Scale bars are 1 mm.

### Composition Optimization and Mechanical Properties

In the exemplary implementation, PEGDA was selected for the base material and a mixture of PEGDA and HAMA was used for the fabrication of succulent-inspired tips. To strike a balance between economical usage of printing materials and optimal printability deriving from the high concentration of these materials, printability across various HAMA and PEGDA mixture compositions was assessed. FIG. 4 displays the results of this evaluation as a matrix of HAMA content versus PEGDA content. Mixtures falling within the area to the right of the dashed line were deemed most printable while the lightly shaded area to the left side of the graph were considered non-printable. On average, an HAMA concentration greater than 5% and a PEGDA concentration greater than 7.5% (black dot at center) were considered to provide robust printing.

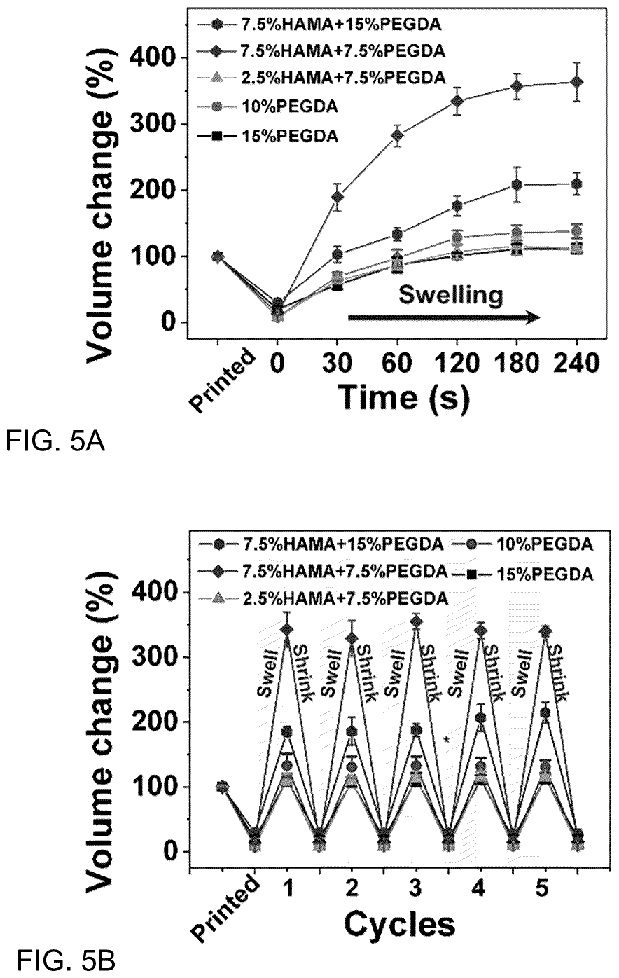

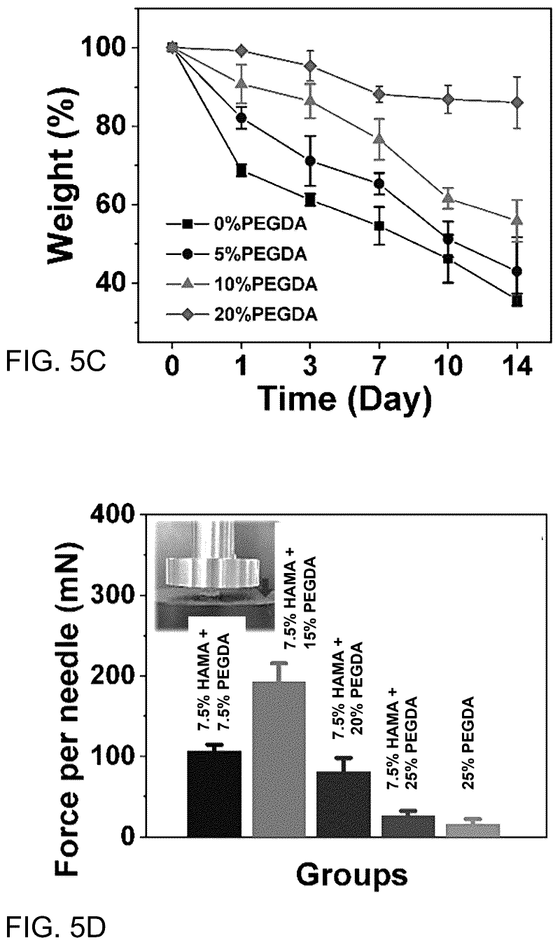

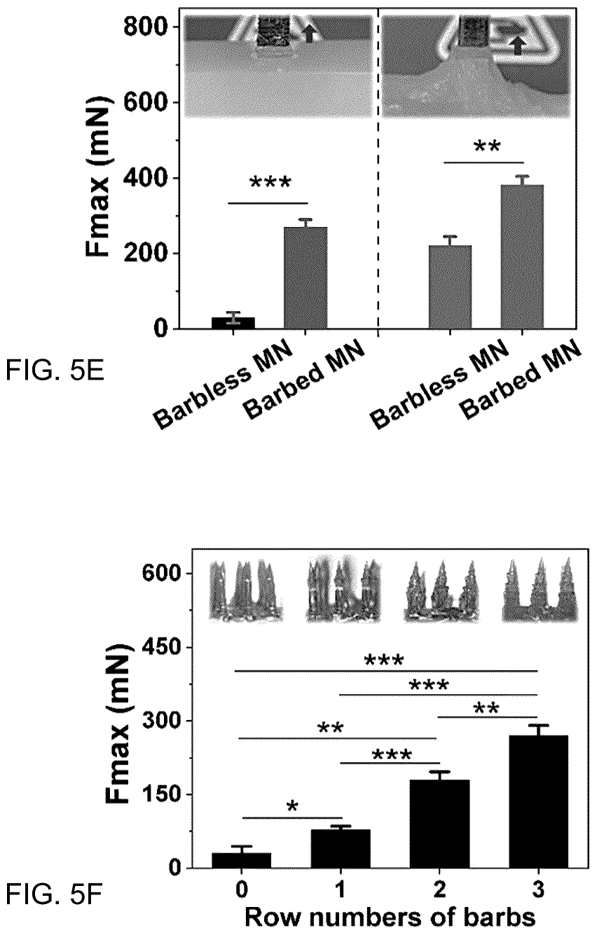

For fabrication of embodiments of the MN tip array with succulent-like water sensitive ability, a relatively large volume change ratio responding to external water content is desirable. This characteristic enhances adhesion within the skin tissue via the swelling of the barbs and allows for the formation of a sharp, stiff tip for skin penetration through dehydration. MN patches with different HAMA and PEGDA concentrations were treated to induce dehydration and rehydration to evaluate their volume change ratio. As shown in FIG. 5A, volume changes were measured over a period from 0 seconds (dehydrated) to 240 seconds. MN patches from all groups displayed the ability to shrink after dehydration and revert to their original sizes upon water absorption. It was observed that a higher HAMA concentration resulted in a higher swelling ratio, while the elevation of PEGDA concentrations showed an opposite effect. Based on these results, a mixture of 7.5% HAMA and 7.5% PEGDA (upper curve in FIG. 5A) was found to exhibit the most pronounced volume change between dehydrated and rehydrated statues.

Multiple swell-shrink cycles were induced to further explore the reversible shrink-swell behavior of the MN patches in response to external humidity (FIG. 5B). MN patches with different HAMA and PEGDA compositions were dehydrated and rehydrated for five cycles. All compositions showed consistent reversibility, retaining their dimensions in both dehydrated and hydrated conditions. This consistency suggests the feasibility of efficient drug loading by immersing the MN patches in a drug solution, followed by their potential to revert to a sharp tip for skin penetration. Although a higher HAMA concentration could enhance volume change, precursor solutions with a HAMA concentration exceeding 7.5% was determined to become too viscous, thus impeding the printing of MN patch with succulent-like microstructures. Thus, 7.5% was identified as the optimal HAMA concentration and used for printing in subsequent experiments.

Concerning the PEGDA concentration, the addition of PEGDA to HAMA could enhance printability and the degree of crosslinking but hampers the biodegradability of the MN patches. To simulate in vivo degradation conditions, MN patches manufactured with a constant HAMA concentration of 7.5% and varying PEGDA concentrations were immersed in PBS with hyaluronidase, and the weight loss was monitored for 14 days (FIG. 5C). The pure HAMA group (lowest curve) exhibited a weight reduction of 64.19±1.58% from its original weight, whereas the group with 20% PEGDA (upper curve) showed only 13.95±6.54% reduction. This observation suggests that a higher PEGDA concentration leads to a reduced weight loss, i.e., additional PEGDA network decelerates degradation of MN patches.

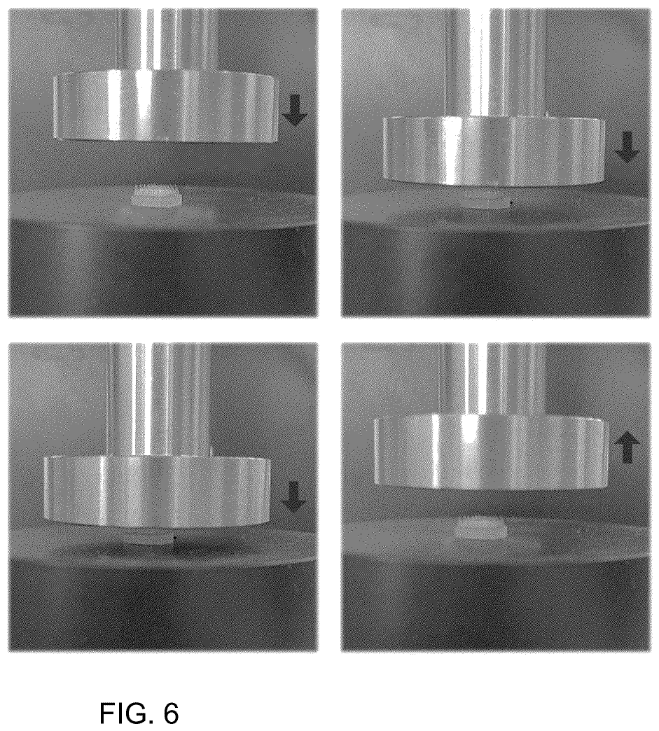

As MN patches experience an increasing compressive force when penetrating into the skin, it is essential that they withstand a tissue-puncturing pressure. A typical compression test was conducted to assess the mechanical properties of the MN patches. Referring to FIG. 6, a MN sample was laid on a platform, where the MN tips pointed to the pressure sensor. The compression force was recorded as the sensor gradually moved to the tips. Arrows indicate the moving direction of the sensor. MN patches of different compositions were positioned on a stage with a platen situated above the sample for compression. The compression force variations were recorded and plotted in FIG. 5D. As shown, MNs formed from 7.5% HA and 15% PEGDA exhibited the highest force value during the compression. In general, MN patches with higher HAMA concentrations exhibited greater stiffness, while those with a higher PEGDA concentration had more flexible MN tips, leading to a reduced compression force per needle. It may be noted that that the force per needle for all the tested groups exceeded 21 mN, which is the minimum required force for skin penetration.

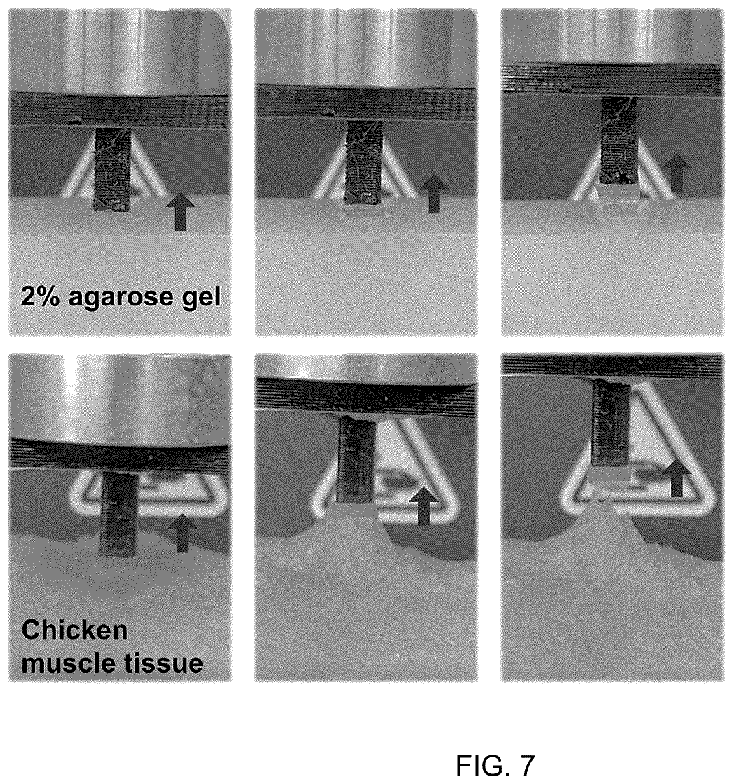

The succulent-inspired extruding barbs should contribute to enhanced adhesion between MN patches and skin tissues. Tensile tests were conducted to assess the adhesion performance of the MN patches. Two different tissue models were employed in the test—agarose gel and chicken breast, representing non-fibrous and fibrous tissues, respectively. (FIG. 7). MN samples were glued to a customized 3D printed probe, with the tissue material fixed on the stage. As shown in FIG. 5E, MN patches were first inserted to the tissue, and then pulled out from the tissue, with the tensile force recorded during the procedure. Both the agarose gel and chicken breast tissue groups showed significant differences between the maximum force of barbless MN patches and barbed MN patches, underscoring the effectiveness of the barb structure in enhancing tissue adhesion.

Additionally, the effect of rows of barbs was examined. Four groups of MN patches were fabricated as shown in FIG. 5F, ranging from no barbs to 3 rows of barbs. Tensile tests were conducted using a chicken breast tissue. It was evident that by increasing the number of rows, the maximum force during the pull-out session could be significantly increased.

### Example 1: Controllable Sustained Drug Release Behavior

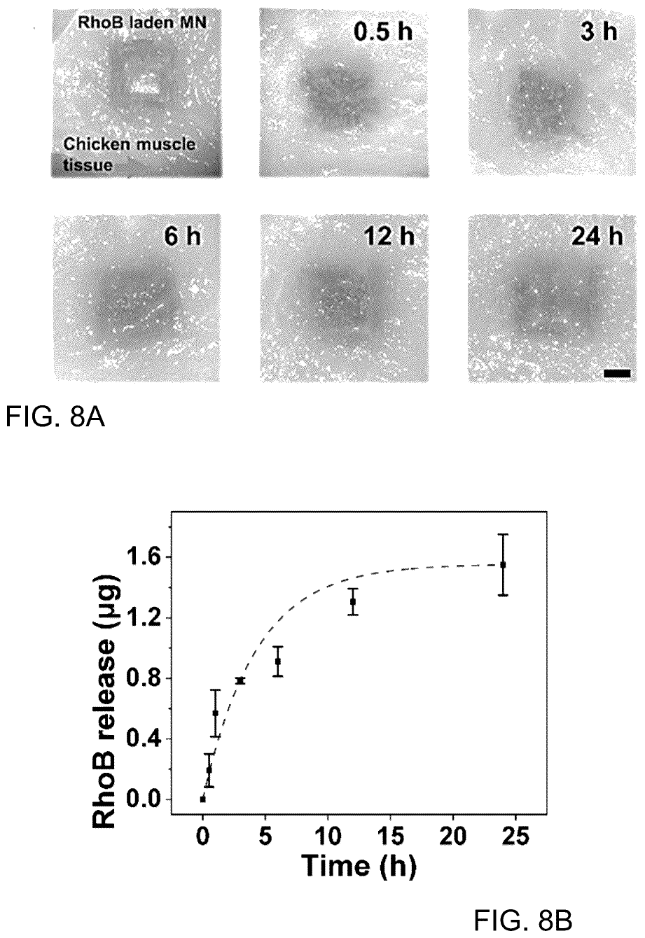

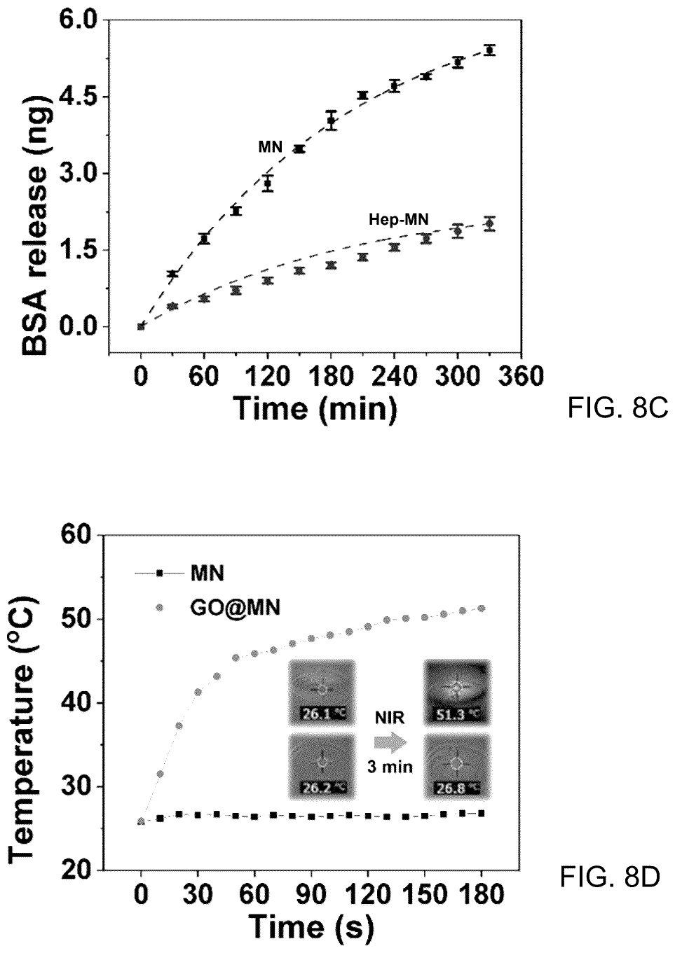

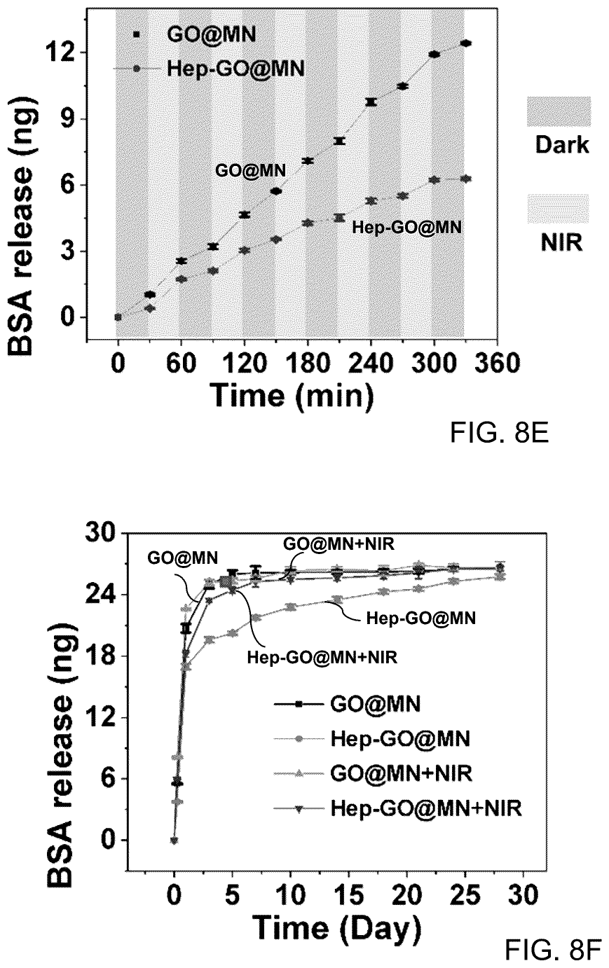

The water-absorbable and porous hydrogels used to form the inventive MN patches allows them to be loaded with a drug through immersion in a drug solution or encapsulation of drug molecules during the printing process. These features facilitate efficient loading of a wide range of drug molecules. For the loading and release test of small molecule drugs, Rhodamine B (RhoB) was employed as the drug model. The dehydrated MN patches were first soaked in a RhoB solution for drug loading during the hydration process. The drug-loaded MN patches were dehydrated again and then inserted into chicken breast tissue for drug release assessment. As shown in FIG. 8A, the RhoB diffusion in chicken breast tissues was tracked over a 24 hour period at time points Oh, 0.5 h, 3 h, 6 h, 12 h and 24 h. By quantifying the RhoB concentration with a spectrophotometer, the drug contents released from the RhoB-loading MN patches was gradually increased over time, and a release amount of 1.55±0.20 μg was achieved in the chicken muscle tissue after 24 hours (FIG. 8B). These results confirm the effectiveness and efficiency of the inventive MN patches in both loading and release of small molecule drugs.

To address the demand for extended drug release, 1% Hep-SH was incorporated into the MN tips. Hep-SH (thiolated heparin), a highly sulfated glycosaminoglycan, possesses a negative charge, which facilitates prolonged release of certain biomolecules through electrostatic interactions. Bovine serum albumin (BSA), a typical drug model of growth factors, was mixed with the precursor solution either with or without Hep-SH prior to printing. BSA release from the fabricated MN patches in PBS was monitored the for 6 hours (360 minutes). The results are plotted in FIG. 8C, with the MN results in the upper curve and the Hep-MN results in the lower curve. As shown, the MN patches containing heparin (i.e., Hep-MN group) demonstrated a more sustained release of BSA compared to those without heparin (i.e., MN group). This observation underscores the ability of Hep-SH to effectively extend the release of growth factors.

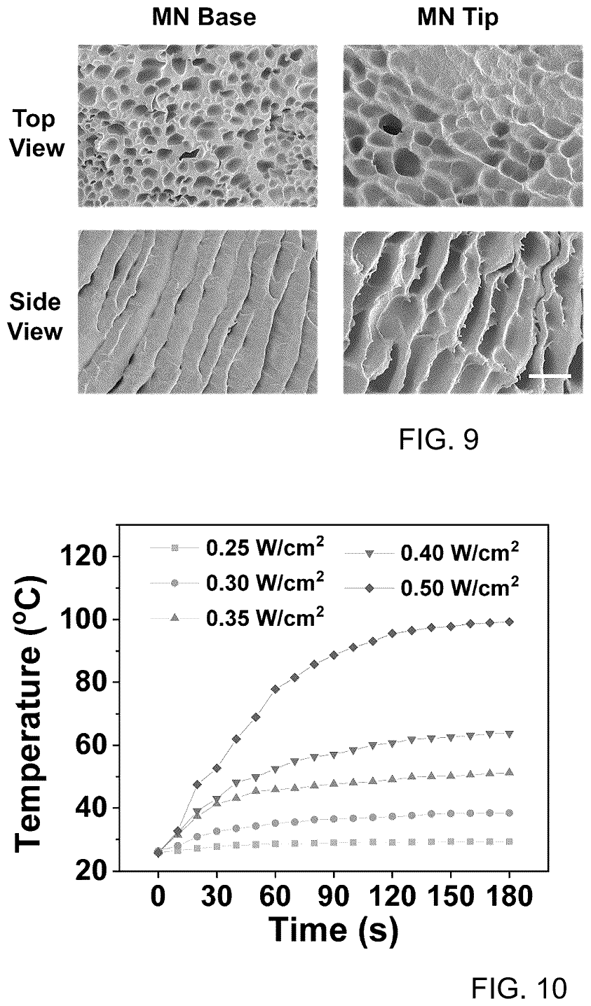

To further evaluate the ability to control drug release with the inventive MN patch, GO (graphene oxide) was incorporated into the MN base to implement a NIR-controlled release. The SEM images in FIG. 9 show the PEGDA/GO base component had small pore sizes with embedded GO sheets, whereas the PEGDA/HAMA mixture of the MN tip component were with larger pore sizes. Due to the photothermal heating ability of GO in the MN base, the temperature of the MN patches could increase under NIR exposure (808 nm). The temperature increase was tracked under NIR exposure with different power densities, showing efficient photo-thermal heating ability of the GO-embedded MN (GO@MN) patches as the temperature could increase to about 100° C. within 180 seconds of NIR exposure of 0.5 W/cm² intensity (FIG. 10). Under moderate NIR intensity 0.35 W/cm², the temperature of GO@MN reached 45.9° C. after NIR irradiation for 60 seconds, whiles the MN patches without GO remained at 26.4° C. under the same irradiation (FIG. 8D). These results demonstrate that embedded GO in the MN base could effectively increase the temperature upon NIR exposure.

Both Hep-SH and GO were incorporated into the inventive MN patches (Hep-GO@MN) to enable a photo-responsive drug release. BSA release from GO@MN and Hep-GO@MN was monitored for 6 hours, employing alternating cycles of 30-min NIR ON (NIR) and 30-min NIR OFF (Dark), indicated by the alternating dark and light gray areas in FIG. 8E. Hep-GO@MN exhibited a slower release rate in comparison to GO@MN, and in both groups, BSA release was expedited under NIR exposure. This observation underscores the real-time responsiveness and acceleration of drug release that can be achieved by integrating GO into the inventive MN patches.

Additionally, the long-term drug release profiles of four distinct groups were investigated over the course of 28 days: GO@MN, Hep-GO@MN, GO@MN+NIR, and Hep-GO@MN+NIR. The groups subjected to NIR exposure were placed under 30 minutes of NIR exposure daily. As shown in FIG. 8F, leveraging the photothermal effect generated by GO, the GO@MN group exposed to NIR exhibited the most rapid release during the initial stages. In contrast, the Hep-GO@MN group maintained a prolonged release profile, indicating a NIR-controlled long-term release capability of the growth factors.

The above-described drug models that were employed to evaluate the efficiency and effectiveness of the controlled and sustained release characteristics of the inventive MN patches are not intended to be limiting As will be appreciated by those in the art, a wide range of biological, small molecule drugs, and growth factors are desirable targets for incorporation in sustained-release delivery systems and are expected to be compatible with the inventive approach to transdermal drug delivery.

### Example 2: In Vitro Biocompatibility and Bioactivity

Cytocompatibility remains an essential requirement for the practical application of the inventive MN patches. Given the prevalent use of transdermal drug release via MN for wound healing, vascular endothelial growth factor (VEGF) was selected as the model drug in this study. VEGF plays a pivotal role in modulating cell proliferation, migration, and angiogenesis, all of which are integral stages in the wound healing process. Thus, VEGF loaded MN patches are expected to facilitate Human Umbilical Vein Endothelial Cells (HUVECs) to proliferate, migrate and form vessel-like networks.

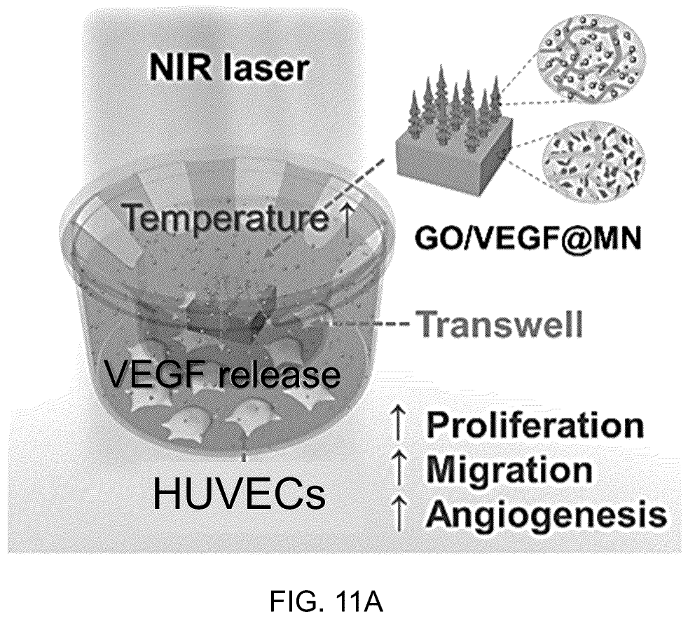

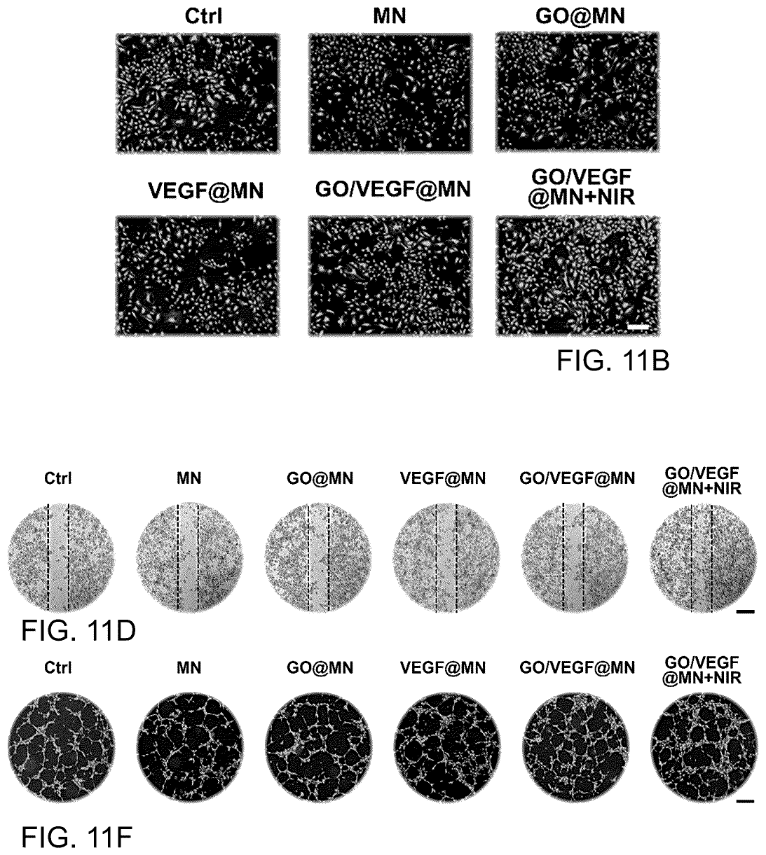

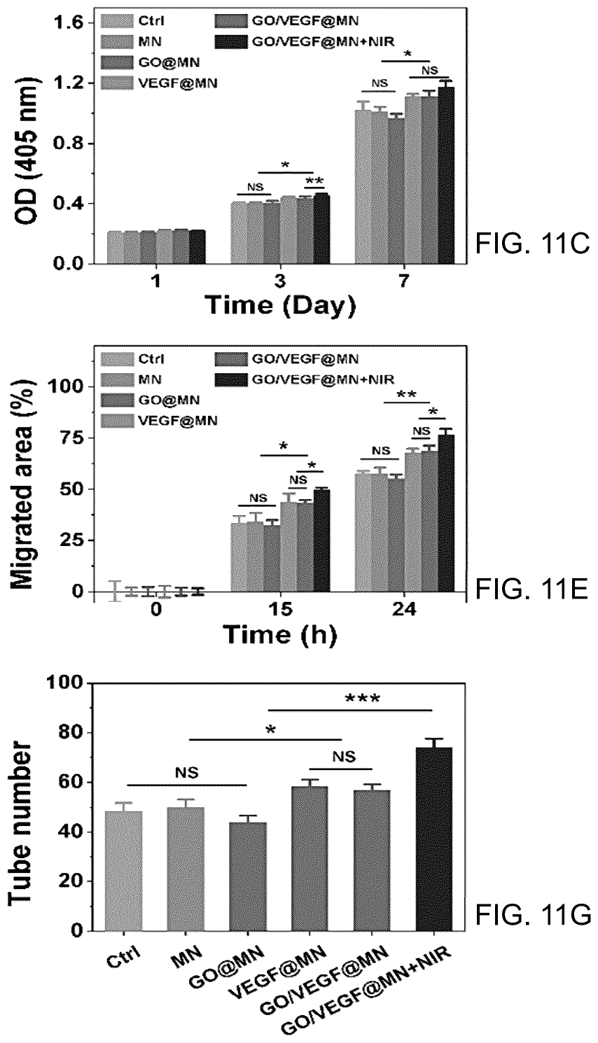

To evaluate the cytocompatibility, MN patches were co-cultured with HUVECs for 7 days. HUVECs were seeded in the bottom of a 24-well plate, while the MN patches were positioned in a TRANSWELL® permeable plate above the HUVECs cells (FIG. 11A). Referring to FIG. 11B, six different groups were designed to examine the cytocompatibility: Control (Ctrl), MN, GO@MN, VEGF@MN, GO/VEGF@MN, GO/VEGF@MN+NIR. (Scale bar=300 μm.) For the GO/VEGF@MN+NIR group, samples were exposed to NIR radiation for 30 min per day. Live Calcein AM staining on day 3 of the culture showed all MN groups did not affect the cell viability in comparison to the control group, and the GO/VEGF@MN+NIR group (lower right panel) showed denser cells as compared to the control group (upper left panel). CCK-8 assay was used to quantify the cell viability of 7-day culture (FIG. 11C), validating that MN, GO or NIR had no noticeable cytotoxicity to HUVECs while VEGF-loading MN groups could promote the cell proliferation. These observations agreed well with earlier studies showing that the MN patches with a photothermal conversion effect could accelerate VEGF release upon NIR irradiation. (See, Sun, L., et al., Multifunctional Microneedle Patches with Aligned Carbon Nanotube Sheet Basement for Promoting Wound Healing, Chem. Eng. J. 2023, 457, 141206.)

The effect of VEGF-loaded MN patches on cell migration was evaluated using a typical scratch assay. HUVECs were uniformly seeded in well plates, and a scratch was created in the center of each well. FIG. 11D shows the different cell groups stained by crystal violet staining after 24 hours. While the control group (left) displayed a clear scratch, the groups with VEGF showed more obvious cell migration to cover the scratch, especially the GO/VEGF@MN+NIR (right) group with the scratch almost fully covered. Quantification of the migrated area revealed that the MN patch groups containing VEGF significantly enhanced cell migration as compared to those without VEGF. Furthermore, the GO/VEGF@MN+NIR group demonstrated a significant improvement in cell migration as compared to groups that contained VEGF but did not undergo NIR exposure (FIG. 11E). These results suggest that VEGF-loaded MN patches could release VEGF during the co-culture to enhance the cell immigration. Notably, the rate of release can be further augmented by NIR exposure.

Furthermore, to assess the proangiogenic effect using a tube formation assay, the MN patches were co-cultured with HUVECs seeded on Matrigel coating. Given the well-known ability of VEGF to promote tube formation, it was evident that groups containing VEGF exhibited more fully formed tubular structures (FIG. 11F). Quantitative analysis of tube numbers revealed that all VEGF-infused groups displayed significantly more tubular structures in comparison to the groups lacking VEGF. Notably, exposure to NIR radiation further enhanced the tube formation (FIG. 11G). The results indicate the ability of VEGF-loaded MN patches to efficiently release VEGF, consequently enhancing proangiogenesis. The combination of VEGF release and NIR exposure synergistically contributed to the enhanced proangiogenesis.

### Example 3: Materials and Methodology

Chemicals: Poly(ethylene) glycol diacrylate (PEGDA, Mn=700 Da), methacrylic anhydride (MA), tartrazine, albumin-fluorescein isothiocyanate conjugate (FITC-BSA), cysteamine, 2-isocyanatocthyl methacrylate, tris(2-carboxyethyl) phosphine hydrochloride (TCEP), heparin sodium salt, and graphene oxide (GO) nanosheets were purchased from Sigma-Aldrich. Lithium phenyl-2,4,6 trimethylbenzoylphosphinate (LAP) was purchased from Tokyo Chemical Industry. Hyaluronic acid (HA, 10 kDa˜1.0 MDa) was purchased from Bloomage Biotechnology. Agarose were purchased from J. T. Baker. Spermine was purchased from Alfa Aesar. 1-Ethyl-3-[3-(dimethylamino) propyl] carbodiimide (EDC), sodium chloride, Ellman's test kit, crystal violet, and rhodamine B (RhoB) were purchased from Thermo Scientific. 1-Hydroxybenzotriazole monohydrate (HOBt) was bought from Tokyo Chemical. Dialysis tubes (10 000 MWCO) were purchased from Spectrum Laboratories. Phosphate-buffered saline (PBS, pH 7.4) was bought from Gibco. Endothelial cell medium (ECM) was purchased from ScienCell. All materials were used as received. Deionized water (18.2 MΩ/cm) was obtained from a Milli-Q water purification system.

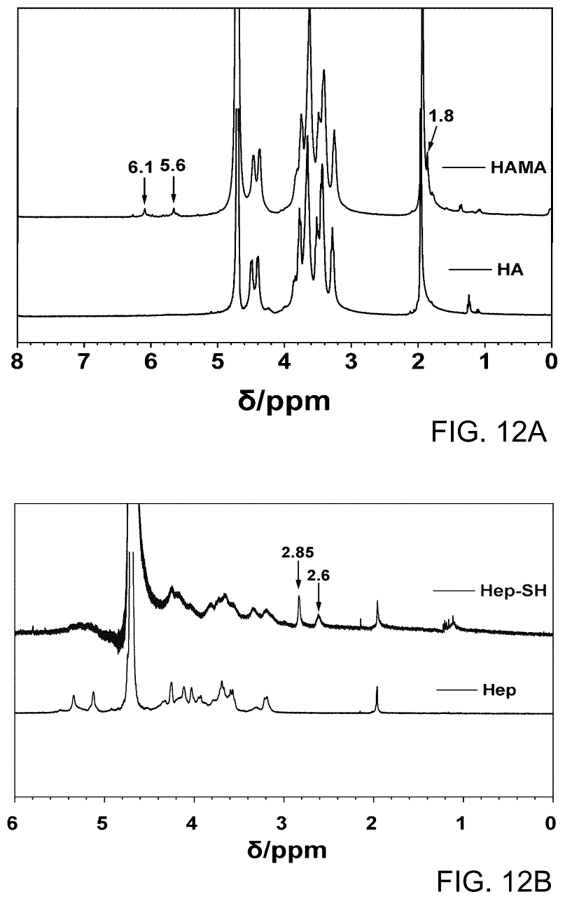

Synthesis of HAMA: Methacrylated hyaluronic acid (HAMA) was synthesized as described by Wu, X., et al., Stem Cell Niche-Inspired Microcarriers with ADSCs Encapsulation for Diabetic Wound Treatment, Bioact. Mater. 2023, 26, 159-168. Briefly, HA (4 g) was dissolved in PBS (200 mL) and continuously stirred for 2 h. Methacrylic anhydride (15 mL) was then added dropwise into the solution while ensuring the pH remains 8˜9 by using 1M NaOH. After stirring at 0° C. for 24 h, the solution was dialyzed against deionized water at room temperature for 7 days, with water replacement every 12 h. After dialysis, HAMA solutions were lyophilized and stored at −80° C. before use. The reagent and product were examined by proton nuclear magnetic resonance (1H NMR, Bruker) to confirm the methacrylation (FIG. 12A).

Synthesis of Hep-SH: The thiolated heparin (Hep-SH) was synthesized as described by Wang, P., et al., Controlled Growth Factor Release in 3D-Printed Hydrogels, Adv. Healthc. Mater. 2020, 9 (15), 1900977. Typically, heparin (200 mg) was dissolved in deionized water (40 mL), and then EDC (91.07 mg) and HOBt (65.05 mg) were added and stirred for 30 min. The pH was adjusted to 6.8 by 5M NaOH, and then cysteamine (78.06 mg) was added. The pH was adjusted to 6.8 by 5M HCl. After reaction at room temperature for 5 h, the solution was dialyzed against deionized water for 24 h, with water replacement every 8 h. The obtained solution was then frozen overnight at −20° C., lyophilized, and stored at −80° C. Subsequently, the lyophilized powder (120 mg) was dissolved in deionized water (40 mL) before adding TCEP (966.7 mg). The pH of the solution was adjusted to 7.5 by 5M NaOH. The neutralization process was kept at room temperature for 1 h. The solution was dialyzed against 5 M NaCl solution with pH adjusted to 5 by 5M HCl for 24 h. The dialysis process was repeated for 3 times. After that, the solution was dialyzed against deionized water for 5 days, with water replacement every 24 h. The final product was then lyophilized and stored in −80° C. before use. The reagent and product were examined by 1H NMR spectra to confirm the thiolation (FIG. 12B).

Printing of MN patches: Prior to 3D printing, the bioinks for printing the MN bases were prepared by mixing the PEGDA prepolymer (15 w/v %) with LAP (0.4 w/v %, a photoinitiator) and tartrazine (0.01 w/v %, a photoabsorber), and the bioinks for printing the MN tips were prepared by mixing the PEGDA (5-25 w/v %) and HAMA (0-10 w/v %) prepolymers with Hep-SH (1 w/v %), LAP (0.4 w/v %) and (0.04 w/v %) tartrazine. The MN patches were fabricated using a home-made DLP-based 3D printing system, which mainly includes precision three-axial stages, an ultraviolet (UV, 385 nm) light source for photopolymerization, a series of optical lenses and a DMD chip for light patterning, and a computer for design and control. (Sec, e.g., U.S. Pat. No. 11,833,742.) During DLP printing, the UV light reflected off the DMD chip, passed through the optical lenses, and polymerized the polymer precursor on the focal plane. The printer's probe was positioned on this focal plane with a methacrylated coverslip attached to its surface, allowing the printed structure to be covalently bonded. As the light pattern projected onto the focal plane, the printer probe ascended at a user-defined speed in a reservoir filled with the photopolymerizable bioinks. After printing the MN bases, another reservoir containing the MN tip bioinks was positioned under the probe, which allowed the construction of the tip portion directly on the base without requiring additional adhesion. For printing GO-incorporated MN patches, the MN base bioinks were supplemented with 1 mg/mL GO nanosheets, and for printing VEGF-incorporated MN samples, the MN tip bioinks were supplemented with 100 ng/mL VEGF. The as-printed MN samples were rinsed with PBS for 3 times, and then dried at 37° C. overnight before use.

Morphological characterizations: The optical bright-field and fluorescent images were obtained using a microscope (Leica DMI 6000B) and a laser confocal microscope (Leica SP8). For microstructure characterization, the hydrogel samples were further lyophilized and their microstructures were observed by a field emission scanning electron microanalyzer (SEM, Zeiss Sigma 500).

Shrinkage and swelling measurement: The MN base samples (cubic: 8 mm×8 mm×2 mm) with different HAMA and PEGDA contents were printed. The length (L), width (W) and height (H) were measured and the volume (V) was calculated according to following equation: V=L×W×H. For swelling measurement, the base samples were soaked in PBS and incubated on a shaker at 37° C. For shrinkage measurement, the remaining water on the sample surface was drained before incubation at 37° C. overnight. The volume was recorded as described above. The volume change ratio of MN base samples was calculated according to following equation: Volume change (%)=Vₜ/V₀×100%. Here, The Vₜ and V₀ represented the volume after and before swelling, respectively.

Mechanical characterizations: Compression and tensile tests of the MN samples were performed on a universal mechanical testing machine (Univert, Cellscale). For the compression force measurement, the MN patches with different HAMA and PEGDA compositions were laid on a platform, where the MN tips pointed to the descending pressure sensor. The sensor gradually moved to the tips at 0.08 mm/s, and the force began to be recorded when touching the MN tips. The force-displacement curves were recorded, and the force at 0.8 mm for different MN samples was recorded. For the adhesion force measurement, two tissue models were used with the agarose gel (2%, 3 cm×3 cm×3 cm) and chicken muscle tissue (3 cm×3 cm×1 cm) as nonfibrous and fibrous tissue models, respectively. The MN sample was fixed on the top customized sample holder using a cyanoacrylate adhesive (e.g., LOCTITE® Super Glue), the tissue models were attached to a bottom fixed plate using an adhesive tape (3M, Maplewood, MN). The MN was lowered down and penetrated into the tissue models up to an insertion depth of 2 mm at a rate of 0.08 mm/s. After 60 seconds of relaxation time, adhesion force was measured as the MN was pulled away from the tissue models up to a displacement of 5 mm. The maximum force for different MN samples were recorded.

In vitro degradability of MN patches: The degradation behavior of MN samples was evaluated based on their weight loss. The samples were immersed in 1 ml of PBS with hyaluronidase (2 U/mL), and shaken at 37° C. At designated time points (on days 0, 1, 3, 7 and 14), the samples were dried completely at 65° C., and their dry weights were recorded. PBS was refreshed at regular intervals. The degradation curve was plotted based on the weight loss.

Release of rhodamine B in chicken muscle tissues: RhoB, as a typical small-molecular drug model, was loaded into the MN samples by immersion in RhoB solution (concentration of 100 μg/mL) for 12 hours. At designated time points (i.e., 0.5, 1, 3, 6, 12, and 24 h), MN patch was removed from the chicken muscle, and the remaining RhoB release in the chicken breast was examined using an optical microscope (Leica). For quantification of RhoB released from the MN samples in the chicken muscle tissues, the RhoB-loaded chicken tissue samples were ground in a mortar for 5 min and 1 mL of DI water was added to obtain a homogeneous mixture. 2 mL of ethanol was then added and the mixture was further ground for 5 min to extract the RhoB from the muscle fibers. Subsequently, the mixture was centrifuged at 1000 rpm for 10 min and the supernatant was collected. The RhoB concentration in the supernatant was determined by measuring the OD value at 554 nm using a microplate reader (Tecan).

Controllable long-term release of BSA in PBS solution: FITC-BSA, as a typical protein drug model, was incorporated into the MN samples by dissolving FITC-BSA (100 ng/mL) in the MN tip bioinks prior to printing. For the FITC-BSA release tests, the BSA-containing MN samples were immersed in PBS and kept shaking at 37° C. For the NIR group, the MN samples were irradiated with an 808-nm laser for 30 min (0.40 W/cm², Temperature˜50° C.) to facilitate the BSA release. At designed time points, the released BSA contents from the MN samples into PBS were quantified based on the FITC fluorescent intensity using a microplate reader (Tecan).

Cell proliferation assay: HUVECs (3˜5 passages) were cultured in complete medium (ECM containing 5% PBS) in a standard cell culture incubator (37° C., 5% CO₂). For the cell proliferation assay, HUVECs (1×10⁴ cells/well) were seeded in a 24-well plate and incubated for 12 h. A TRANSWELL® permeable insert laden with a MN, GO@MN, VEGF@MN, or GO/VEGF@MN patch (GO content: 1 mg/mL, VEGF content: 100 ng/mL) were transferred into each well and incubated with the cells. For the NIR group, the cells were irradiated with an 808-nm laser for 30 min (0.35 W/cm², Temperature˜45° C.) to facilitate the VEGF release every two days. A CCK8 assay was conducted on days 1, 3, and 7. For a typical CCK8 assay, the culture medium was replaced with fresh ECM containing the CCK8 kit (10% v/v). The medium was pipetted after incubation of 2 h, and the absorbance at 450 nm were measured using a microplate reader (Tecan).

In vitro scratch assay: HUVECs (1×10⁵ cells/well) were seeded in a 24-well plate and incubated for 24 h for a confluent monolayer. A sterile 200-μL pipette tip was used to make a single scratch on the cells. Unattached cells were removed by washing the cells twice with FBS free medium and then replaced with ECM containing 1% FBS. A TRANSWELL® insert laden with a MN, GO@MN, VEGF@MN, or GO/VEGF@MN patch (GO content: 1 mg/mL, VEGF content: 100 ng/mL) was transferred into each well and incubated with the cells. For the NIR group, the cells were irradiated with an 808-nm laser for 30 min (temperature˜45° C.) to facilitate the VEGF release. At appropriate time points, the cells were photographed, and the migrated areas were measured using ImageJ software (NIH). The cell migration was quantified as follows: Relative migration area (%)=(1−Wₜ/W₀)×100%. Here, Wₜ represents the wound area at specific time points, while W₀ represents the wound area immediately after scratching.

Tube formation assay: HUVECs (5×10⁴ cells/well) were seeded on a 24-well plate with Matrigel matrix coating (50% v/v in ECM, 250 μL per well). A TRANSWELL® insert laden with a MN, GO@MN, VEGF@MN, or GO/VEGF@MN patch (GO content: 1 mg/mL, VEGF content: 100 ng/ml) was transferred into each well and incubated with the cells for 6 h. For the NIR group, the cells were irradiated with an 808-nm laser for 15 min (Temperature˜45° C.) to facilitate the VEGF release. After removing the TRANSWELL® inserts, the cells were rinsed with PBS, stained with Calcein-AM, and observed under a fluorescence microscope (Leica).

Statistical Analysis: All data were presented as mean±standard deviations. All graphs were generated from OriginPro 2021 software. The statistical significance was evaluated using unpaired Student's t-test between two groups and considered significant when *p<0.05, **p<0.01, and ***p<0.001.

To summarize, DLP printing is used to fabricate succulent-inspired MN patches capable of controllable long-term drug release. The adaptability of the DLP printing process allows for the customization of succulent-like MN tip shapes, accommodating a wide array of practical applications. The fabricated MN patches exhibit excellent shrink-swell volume change and reversibility, ensuring their water sensitive ability and drug loading efficiency. The inventive MN patches possess the robust mechanical properties necessary to penetrate fibrous skin tissue. The improved adhesion force from extruding barbs ensures MN patches remain adhered to the skin tissue for extended drug release. By integrating Hep-SH and GO into the MN matrix, controlled, prolonged drug release can be achieved. The extended release properties of the MNB patches are further enhanced by the ability to accelerate release using NIR exposure. The inventive MN patches exhibit the cytocompatibility essential for practical drug delivery applications, as well as an ability to promote HUVACs cell proliferation, immigration and proangiogenesis. Overall, the inventive MN patches provide significant potential for transdermal therapy and an important solution to the challenge of sustained and controlled drug release.

1. A microneedle (MN) patch, comprising:
a 3-D bioprinted structure comprising:
a substantially planar base formed from a first photocrosslinkable hydrogel material; and
water-sensitive barbed structures formed from a second photocrosslinkable hydrogel material printed onto and extending substantially perpendicular from a surface of the base, the barbed structures each having a volume configured to reversibly shrink and swell in response to surrounding humidity, wherein the barbed structures have a hardness configured to penetrate live skin tissue.

2. The MN patch of claim 1, wherein the first photocrosslinkable hydrogel material comprises a polyethylene glycol diacrylate (PEGDA) gel.

3. The MN patch of claim 1, wherein the second photocrosslinkable hydrogel material comprises a mixture of PEGDA and methacrylated hyaluronic acid (HAMA).

4. The MN of claim 3, wherein the mixture comprises from 2.5% to 7.5% HAMA and from 7.5% to 20% PEGDA.

5. The MN of claim 3, wherein the mixture comprises 7.5% HAMA and 7.5% PEGDA.

6. The MN of claim 1, wherein the base further comprises graphene oxide (GO), wherein the GO is configured to heat in response to exposure to near infrared (NIR) light.

7. The MN of claim 1, wherein the second photocrosslinkable hydrogel material further comprises thiolated heparin (Hep-SH).

8. The MN of claim 1, wherein the second photocrosslinkable hydrogel material further comprises a drug configured for transdermal delivery.

9. The MN patch of claim 1, wherein the barbed structures are printed in layers to define alternating layers of radially-extending barbs along a height of each barbed structure.

10. The MN of claim 1, wherein the first photocrosslinkable hydrogel material and the second photocrosslinkable hydrogel material are configured to polymerize in response to exposure to light within a wavelength range of 365 nm to 405 nm.

11. A microneedle (MN) patch for transdermal drug delivery, comprising:
a substantially planar base 3-D bioprinted from a first bioink solution; and
water-sensitive barbed structures 3-D bioprinted from a second bioink solution onto and extending substantially perpendicular from a surface of the base, the barbed structures each having a volume configured to reversibly shrink and swell in response to surrounding humidity, wherein the barbed structures have a hardness configured to penetrate live skin tissue.

12. The MN patch of claim 11, wherein the first bioink solution comprises a polyethylene glycol diacrylate (PEGDA) gel.

13. The MN patch of claim 11, wherein the second bioink solution comprises a mixture of PEGDA and methacrylated hyaluronic acid (HAMA).

14. The MN of claim 13, wherein the mixture comprises from 2.5% to 7.5% HAMA and from 7.5% to 20% PEGDA.

15. The MN of claim 13, wherein the mixture comprises 7.5% HAMA and 7.5% PEGDA.

16. The MN of claim 11, wherein the first bioink solution further comprises graphene oxide (GO), wherein the GO is configured to heat in response to exposure to near infrared (NIR) light.

17. The MN of claim 11, wherein the second bioink solution further comprises thiolated heparin (Hep-SH).

18. The MN of claim 11, wherein the second bioink solution further comprises a drug configured for transdermal delivery.

19. The MN patch of claim 11, wherein the barbed structures are printed in layers to define alternating layers of radially-extending barbs along a height of each barbed structure.

20. The MN of claim 11, wherein the first bioink solution and the second bioink solution are configured to polymerize in response to exposure to light within a wavelength range of 365 nm to 405 nm.
